(19) United States
(12) Patent Application Publication
(10) Pub. No.: US 2017/0245796 A1
(43) Pub. Date: Aug. 31, 2017

(54) OPTICAL SENSOR DEVICE

(71) Applicant: BAR ILAN UNlVERSITY, Ramat Gan (IL)

(72) Inventors: Zeev ZALEVSKY, Rosh HaAyin (IL); Yevgeny BEIDERMAN, Tel Aviv (IL)

(21) Appl. No.: 15/508,265

(22) PCT Filed: Sep. 3, 2015
(86) PCT No.: PCT/IL2015/050884

§ 371 Date: Mar. 2, 2017

Related U.S. Application Data
(60) Provisional application No. 62/045,579, filed on Sep. 4, 2014.

(51) Int. Cl. A61B 5/00; A61B 5/0205; G02F 1/01; A61B 5/145; A61B 5/026; G01N 21/47; A61B 5/1455

(52) U.S. Cl. CPC A61B 5/6804; G01N 21/47; A61B 5/0205; A61B 5/14551; A61B 5/14546; A61B 5/026; A61B 5/0015; G02F 1/0128; G01B 9/02049

(57) ABSTRACT

An optical sensing system is presented for monitoring one or more parameters or conditions of an object. The optical sensing system comprises: an elongated light guide configured for placing in proximity of the object, the light guide defining a cavity for light propagation therethrough along at least one light propagation path, and having a light input port and at least one light output port; and a detector system for receiving light propagating from the at least one light output port, the detector system being configured and operable for monitoring a signal modulated by light interaction with the object and being indicative of the at least one parameter/condition of the object.

### TECHNOLOGICAL FIELD AND BACKGROUND

This invention is in the field of sensing techniques, and relates to an optical sensing device, suitable for use in various applications, including speech signal detection, as well as medical applications for monitoring various biological parameters and conditions.

Optical sensing techniques for monitoring vibrations originated at an object and detecting the object's conditions, such as speech and various biological parameters have been developed, and are described for example in the following patent publications, all assigned to the assignee of the present application: WO09013738; WO12101644; WO14020611. These techniques are based on imaging coherent speckle patterns propagating from an object/subject (e.g. internal organ) in response to coherent defocused illumination; and determining various conditions of the object (e.g. biological or biochemical conditions of the subject) that affect a motion (vibration) of the respective portion of the object/subject.

### GENERAL DESCRIPTION

There is a need in the art for a novel technique enabling effective monitoring of various object's conditions, by monitoring vibrations originated in at least a part of the object.

The present invention provides a novel optical sensing system of the kind specified, which can be used in various applications. These include data communication, subject's behavior (e.g. speech detection), medical applications (e.g. measurement of biological/physiological parameters), as well as industrial applications (e.g. monitoring the state of vibrations of such structures as buildings, bridges, civil structures, pipes, as well as for pipes leakage monitoring, acoustic signal recovery, earthquake monitoring, detection of underground acoustic wave sources).

The technique of the present invention provides for monitoring/determining various object's conditions, based on optical detection/monitoring of the vibrations/motion originated in at least a part of the object. More specifically, the invention relates to determination of various parameters and conditions of a subject (e.g. human, animal) body, and is therefore exemplified below with respect to this specific application. It should, however, be understood that the invention should not be limited to these specific applications. Therefore, the terms “body”, “tissue”, as well as “subject”, used in the description below should be interpreted broadly, i.e. “body” and/or “tissue” constituting a part of an “object” being monitored/inspected.

Thus, the present invention utilizes optical detection of the motion of an object, such as the subject's body/tissue, for identifying one or more parameters/conditions of the object. To this end, the invention provides an optical sensor, which may be implemented as a fiber-based optical sensor. It should be understood that the term “fiber” used herein actually refers to an elongated light guide. As will be described more specifically further below, in some embodiments/applications of the invention, a physical contact between the fiber-based sensor and the body is needed, and in some embodiments contactless detection is performed while in a close proximity to the body.

In some embodiments, the optical sensor of the invention is a two-part structure, where one part is the fiber part, which performs the sensing itself, and the other part is a connector including an electronic circuit (e.g. processing unit and/or power supply (battery), and/or wireless transmission unit, and/or light source and/or a detector). This allows the fiber part (and an accessory into which the fiber part is embedded, as the case may be) to be disposable, while putting the connector in a place in which it is re-usable.

The invention is based on monitoring the body motion via identification of modulation profile over time (during a measurement session) in the detected light response of the body. It should be understood that the term “light response” used herein refers to light returned from the illuminated portion of body and being modulated by interaction with the body. Such “modulation” may be an amplitude modulation, or a change in a polarization state of detected light, or a change in an interference effect/pattern.

In different embodiments of the invention, light modulated by interaction with the body may be light directly interacting with the body, or via a deformation of a light guide through which the light passes (i.e. change of the optical path of light passing through the light guide). In the latter case, the light guide is substantially flexible and is positioned in direct contact with the body during the monitoring session. Thus, for some embodiments, the light guide is to be flexible, and for some other embodiments it needs not to be flexible.

It should also be understood that the detected interference may be interference between a light beam modulated by the interaction (directly or not) with the body and a light beam having no such modulation, e.g. a reference light beam, or this may be interference between portions of the same light beam before and after direct interaction with the body (a so-called “self-interference”). Such self-interference may for example be interference of light components back reflected from two different sections/locations along the light guide (e.g. optical fiber), where one section/location is affected by an external signal originated at the object being monitored (generating movement or deformation of that section of the fiber) and the other section is not.

Thus, the present invention provides an optical sensor including an elongated light guide (e.g. optical fiber), having a light input port (e.g. at one end of the light guide) and a light output/collection port associated with a detector system (e.g. interferometric detector system). The light input and output ports may be at the same or opposite ends of the light guide. The light guide defines a path for light propagation therethrough between the light input and output ports.

In some embodiments, e.g. where direct interaction between light and body is considered, the light guide is formed with one or more so-called “light interaction ports” arranged at one or more locations along a portion of the light guide in between the light input and output ports (e.g. between its opposite ends). The light interaction port is actually a light input/output port (e.g. perforation, defect) through which light emerges from the light guide towards the body and, after interacting with the body, returns back into the light guide. This light is indicative of the light response of the body. In such embodiments, the optical sensor system may include one or more internal light directors arranged inside the light guide for re-directing/deflecting light propagating through the light guide towards the light output port associated with the interferometric detector, i.e. where the interferometric detector is located or where the light is collected and directed to the interferometric detector (e.g. via an external light guide). Also, the optical sensor system may include external light director(s) (e.g. at the outer surface of the light guide) for directing light returned from the body back into the light guide through the respective light interacting port(s).

As indicated above, in some embodiments, the interference detection is used. The interferometric based detection technique may utilize a reference beam, in which case the interferometric detector system is equipped with a typical optics for controllably affecting a change in the optical path of the reference beam, in a conventional manner. If the self-interference is considered, then there is no need for such equipment.

Further, in some embodiments, input light used in the system is previously modulated using a certain known modulating function, to thereby increase SNR of detection. Also, in some embodiments, the input light may include a set of different wavelengths and/or polarization states, which may for example be advantageous when different parameters/conditions of the body are to be detected in the same measurement session. The detection according to which the external signal is obtained (i.e. interaction with the object) can include sensing of the change in the phase (detected by interference effect/pattern), the amplitude modulation induced by the interaction, as well as the change of polarization of light back reflected from the light guide module.

In some embodiments, the light guide includes more than one light guiding elements defining a corresponding number of light propagation paths respectively. For example, the light guide may include an array of light guiding elements, e.g. optical fibers. In some embodiments, each such light guiding element is associated with its interferometric detector, and all are associated with the common control unit (processor). This may be used for concurrently performing multiple measurement sessions with respect to the same parameter and thus improving the accuracy of measurements, or for concurrently measuring several different parameters. Also, in some embodiments, the use of several light guiding elements (e.g. several fibers, e.g. integrated in the fabric warned by individual) in different spatial locations provides for analyzing the time of arrival of the modulated signal to each one of the light guiding elements and extracting the blood flow velocity and volume in the individual.

Thus, according to one broad aspect of the invention, there is provided an optical sensing system for monitoring one or more parameters or conditions of an object, the optical sensing system comprising: a light guide unit comprising at least one elongated light guide configured for placing in proximity of the object, the light guide defining a cavity for light propagation therethrough along a light propagation path, and having a light input port and at least one light output port; and a detector system for receiving light propagating from the at least one light output port, the detector system being configured and operable for monitoring a signal modulated by light interaction with the object and being indicative of the at least one parameter/condition of the object.

It should be noted that the term “proximity of the object” used herein with respect to a location of the light propagation path actually refers to the light guide location in a close proximity to the object or in physical contact with the object.

According to another broad aspect of the invention, there is provided an optical sensing system for monitoring one or more parameters or conditions of an object, the optical sensing system comprising: a light guide unit comprising at least one elongated light guide configured for placing in proximity of the object, the light guide defining a cavity for light propagation therethrough along a light propagation path, and having a light input port and at least one light output port; and an interferometric detector for receiving light from the at least one light output port, and configured and operable for monitoring an interference signal resulting from interference between the collected light, being modulated by interaction with the object, and non-modulated light, the interference signal being indicative of the at least one parameter/condition of the object.

According to yet another broad aspect of the invention, there is provided an optical sensing system for use in monitoring one or more parameters of an object, the optical sensing system comprising: a light guide unit comprising at least one elongated light guide configured for placing in proximity of the object, the light guide defining a cavity for light propagation therethrough along a light propagation path, the light guide having a light input port for inputting light to propagate along said light propagation path, and one or more interaction ports downstream of the input port, the interaction port being configured to allow light propagating inside the light guide to emerge from the light guide towards the object and receive light returned from the object and being modulated by interaction with the object; and a communication utility for transmitting light modulated by interaction with the object to a detector system for monitoring a signal modulated by light interaction with the object and being indicative of the at least one parameter/condition of the object.

For example, the detector system is configured as an interferometric detector system for monitoring an interference signal resulting from interference between the modulated light and non-modulated light, the interference signal being indicative of the at least one parameter of the object.

According to yet further aspect of the invention, it provides an optical sensing system for monitoring one or more parameters or conditions of an object comprising: a light guide unit comprising at least one elongated light guide configured for placing in proximity of the object, the light guide defining a cavity for light propagation therethrough along a light propagation path, and having a light input port for inputting light to propagate along said light propagation path, and one or more interaction ports downstream of the input port, the interaction port being configured to allow light propagating inside the light guide to emerge from the light guide towards the object and receive light propagating from the object and being modulated by interaction with the object; and a detector system (e.g. an interferometric detector system) configured and operable for detecting light output from the light guide and monitoring a signal modulated by light interaction with the object (e.g. an interference signal resulting from interference between the light modulated by the interaction with the object and non-modulated light), being indicative of at least one parameter/condition of the body.

According to yet further aspect of the invention, there is provided an optical sensing system for monitoring one or more parameters or conditions of an object comprising: a light guide unit comprising at least one elongated flexible light guide configured for placing in contact with the object, the light guide defining a cavity for light propagation therethrough along a light propagation path, and having light input and output ports at the same or opposite ends of the light guide, interaction between the flexible light guide and the object causing deformation and/or movement of the light guide according to a movement originated at the object, thereby modulating light propagating along said light propagation path; and a detector system (e.g. an interferometric detector system) for receiving light from the light output port, and configured and operable for monitoring a signal modulated by light interaction with the object (e.g. interference signal resulting from interference between the modulated light and non-modulated light), being indicative of at least one parameter/condition of the object causing said movement of the object.

As indicated above, the light guide may be formed by an optical fiber, which may be very thin and desirable flexible thus allowing its use in various applications. For example, such fiber(s) may be used in a fabric material.

The present invention also provides an optical sensor device for use in an optical sensing system for monitoring one or more parameters of an object. The sensor device comprises a light guide unit comprising at least one elongated flexible light guide configured for placing in contact with the object, the light guide defining a cavity for light propagation therethrough along a light propagation path, and having light input and output ports at the same or opposite ends of the light guide, interaction between the flexible light guide and the object causing deformation of the light guide according to a movement originated at the object, thereby modulating light propagating along said light propagation path, such that a modulation pattern corresponds to a motion pattern of the object, being thereby indicative of one or more parameters of the object.

In yet further aspect of the invention, it provides an optical sensor device for use in an optical sensing system for monitoring one or more parameters of an object, the sensor device comprising a light guide unit comprising at least one elongated light guide configured for placing in proximity of the object, the light guide defining a cavity for light propagation therethrough along a light propagation path, and having a light input port for inputting light to propagate along said path, and one or more interaction ports downstream of the input port, the interaction port being configured to allow light propagating inside the light guide to emerge from the light guide towards the object and receive light returned from the object and being modulated by a modulation pattern indicative of the interaction of light with the object, said modulation pattern corresponding to a motion pattern of the object and being thereby indicative of one or more parameters of the object.

Generally, the optical sensor system includes a fiber part including the optical sensor device configured as described above (i.e. including light input and output ports, and possible also light interaction port(s)); and a connector part including an electronic circuit (processing unit and/or battery and/or wireless transmission unit), and possible also a light source and a detector. The fiber part may be disposable, possible also together with an accessory in which it is embedded (e.g. fabric), while the connector part may be reusable or may also be disposable. For example, in applications where the optical sensor system is intended for a short term use, the connector part may be configured to be disposable as well. Such disposable connector part may for example utilize a capacitor based circuit, instead of the conventional power supply unit, and configured for transmitting the measured data (condition/status of the object being monitored) in a less frequent manner (e.g. every hour). This may significantly reduce the costs of the connector part and thus allow it to become disposable.

Possible applications in which the disposable feature is relevant include diapers, where the use of the sensing device for a short period of time is also very relevant.

It should be noted that the optical sensor system may be used with cloths related applications (shirts, shoes, etc), as well as sheets, hats, buttons, bras, underwear, belts and even jewelries, by embedding the fiber part therein. The optical sensor system (at least the fiber part thereof) can be integrated into swimming suits and allow monitoring of bio-medical parameters also under water or in wet environment.

The invention also provides a fabric material carrying the above described optical sensing system or at least the above-described optical sensor device (e.g. the optical sensing system or at least the optical sensor device being embedded in the fabric material). Such a fabric material equipped with the sensing technology of the invention, may be configured for non-contact bio monitor of various parameters including for example breathing, heart beating or any other biomedical parameters such as blood pulse pressure, blood oxymetry related parameters. Considering bio monitoring of the pulse oxymetry related parameter(s), a ratio between the modulated signals for two wavelengths is determined, one red light of 600-750 nm wavelength light band and one infrared light being in the 850-1000 nm wavelength band, Also, the invention can be used for non-contact bio monitoring of lactate concentration. To this end, the fiber sensing unit is positioned within the fabric to be warned by woman at a location close to the muscle in which the concentration is aimed to be sensed.

The invention also provides for determining blood flow velocity and volume. To this end, several light guides (fibers) are used being integrated in the fabric in different spatial locations; the time of the arriving modulated signal to each one of them is analyzed in order to extract the blood flow velocity and volume. For example, two fiber sensors may be used positioned in a spaced-apart relationship (a few centimeters apart) along the same blood artery, and the light propagation in each for fibers is used measuring the heart beating pulse. A time difference between the heart beating pulse measured by the two spaced-apart fibers (i.e. the time the same heart beat progresses from the location of one fiber to that of the other) allows for extracting the blood flow velocity by computing the ratio of the distance between the two fibers and the time the same heart beat is measured at each one of them.

Further, the invention can be used for recording acoustic signals indicative of conversations being performed by a wearer of said fabric material and in a range of up to a few meters from the wearer.

As indicated above, the invention also provides a capability of monitoring liquid leakage out of pipes, while the light guide is installed nearby the pipes.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a block diagram of an optical sensing system of the invention;

FIGS. 2A and 2B are schematic illustrations of the configuration and operation of an optical sensing system according to some embodiments of the invention;

FIGS. 3A to 3D illustrate a specific example of the optical sensing system of the invention, where FIG. 3A shows a basic optical setup, FIG. 3B shows a snapshot of the basic experimental system; and FIGS. 3C and 3D present two alternatives for usage/integration of the fiber based optical sensor into the fabric; and

FIGS. 4A to 4C illustrate experimental results for bio-medical non-contact fiber based sensing system of the invention, where FIG. 4A shows a periodic signal of heart beats taken from T-Shirt with fiber based sensor, FIG. 4B shows extraction of breathing recorded via the fiber positioned near the chest of a subject, and FIG. 4C shows sound extracted with the fiber-microphone installation.

### DETAILED DESCRIPTION OF EMBODIMENTS

The present invention provides a novel optical sensor device and a sensing system using the same, in which one or more parameters of an object (e.g. subject's body) are determined based on monitoring light propagation through a light guide located in the proximity of the object (close proximity or in physical contact with the object), such that the detected light is indicative of/modulated by interaction between the object and either the light from the light guide or the light guide itself. In other words, the modulation of the detected light is a result of either direct interaction between the light and object, or via deformation of the light guide (change of the optical path of light in the light guide) as a result of direct interaction between the light guide and object.

The optical sensor device of the invention includes an elongated light guide (e.g. optical fiber) defining a path for light propagation therethrough, and having a light input port for receiving input light from a light source (e.g. laser), and a light output port where light is collected towards a detector system. The light input and output ports may be at the same or opposite ends of the light guide.

Thus, in some embodiments of the invention, a fiber is used as a light guide, which can be a plastic fiber rather than glass one (e.g. to make it more flexible when integrated into a fabric). In some embodiments, light redirecting elements (at times termed here “scattering points”) may be provided in the fiber core arranged in a spaced-apart relationship along the axial dimension, i.e. along the light propagation path. In the embodiments where the light input and the light output ports are associated with the same location (end portion) of the fiber, the scattering points may be used to cause the light to be back reflected to propagate back towards the input/output end of the fiber, which in some embodiments may also be used for monitoring the self-interference between these light components. Alternatively or additionally, the scattering points may be used as interaction ports to cause light to exit the fiber, interact with the nearby tissue and be back reflected from it and coupled back into the fiber. The scattering points may be discontinuity points along the core (cavity of light propagation) of the elongated light guide. The discontinuity may be due to change in the real or imaginary parts of the refraction index of the light guiding core.

The light that is back reflected is detected, e.g. via interference with the injected (input) light beam, and used to extract temporal changes in the detected light. Generally, the inventors have shown that temporal changes in the detected light, corresponding to light modulation by interaction with an object being monitored (direct interaction or via deformation of the light guide), can be used as a “microphone” at proximity of the object, e.g. embedded into the fabric, or as an embedded biomedical/biometric sensor. The inventors have experimentally demonstrated the capability of the optical sensing system to “hear” sound (voices), as well as to sense heart beating and breathing, without having full contact between the fiber and the measured subject. The fiber sensors can be incorporated into the fabric (shirt, sheets, shoes etc) and used for biomechanical sensing (breathing of babies when incorporated into sheets), biomedical monitoring for subjects (heart beats), biochemical monitoring for subjects (for instance, alcohol level in blood). The fabricated fibers can be thinner than the fibers used for optic communication (since for the purposes of the invention there is no need to conduct light for distances of hundreds of kilometers) and can be as thin as only few tens of microns.

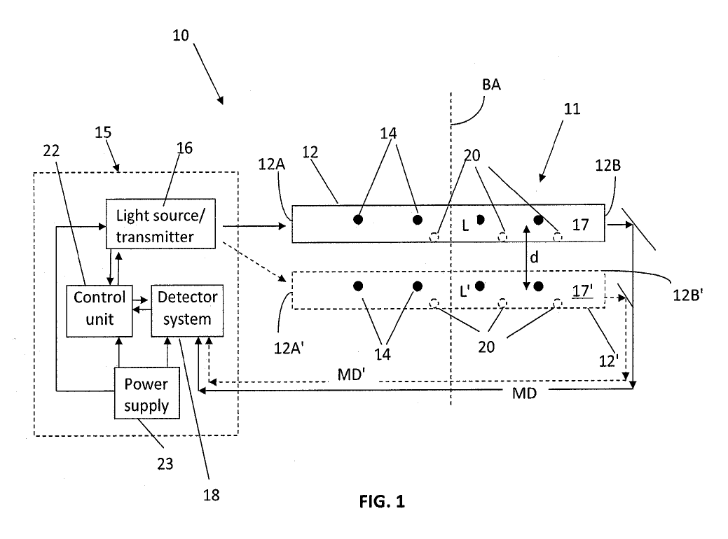

FIG. 1 schematically illustrates, by way of a block diagram, an optical sensing system 10 of the invention for monitoring one or more parameters/conditions of an object 30. The optical sensing system 10 includes an optical sensor device 11 (a so-called “fiber part” of the system 10) formed by an elongated light guide 12 configured for placing in proximity of an object to be monitored, and an electronic unit 15 (a so-called “connector” part of the system 10). The light guide unit 12 defines at least one cavity 17 for light propagation therethrough along at least one light propagation path. Generally speaking, the light guide unit 12 includes one or more light guides (at times referred to as light guiding elements) each defining a light propagation path. The light guide has a light input port 12A and one or more light output ports 12B. In this schematic illustration, the light input and output ports 12A and 12B are exemplified as being associated with opposite ends of the light guide. It should, however, be understood, and will also be exemplified further below, that the invention is not limited to this configuration.

In some embodiments, the optical sensor device 11 may also include one or more interaction ports, generally at 20, located inside the light guide downstream of the input port 12A. The interaction port 20 is actually a light input/output port configured to allow light propagating in the cavity 17 of the light guide 12 to emerge from the light guide towards the object 30 and receive and light returned from the object 30 and being modulated by direct interaction with the object. The provision of the interaction port(s) is optional, and is used in the device configuration utilizing direct interaction between the light and object.

In some embodiments, the optical sensor device 10 also includes one or more internal light redirecting elements, generally at 14, located inside the light guide and being arranged in a spaced-apart relationship along the light propagation path. The light redirecting elements 14 reflect/deflect light propagating inside the cavity towards the light output 12B.

The electronic unit 15 includes a control unit (processor unit) 22 and a power supply unit (battery) 23, and may also include or be connectable to a light source/transmitter unit 16 and a detector system 18. As shown, light from the light source 16 is input to the light guide 12 via the light input port 12A, and light is collected at the output port 12B to be received by the detector system 18, either directly by locating the detector at the light output port 12B or via suitable light directing element(s) such a light guide, mirror(s), etc.

The light source unit 22 may be configured for producing single- or multi-wavelength input light, and/or light polarized light. The detection system is configured and operable for receiving output light and generating measured data indicative thereof. As will be described more specifically further below, the detected light is indicative of (modulated by) light interaction with the object. The control unit 22 is configured for receiving measured data and processing it to identify the modulation and determine one or more parameters/conditions of the object.

As further schematically shown in FIG. 1, in some embodiments, the light guide unit 12 may include at least one additional light guide 12′ having input and output ports 12A′ and 12B′. The two light guides 12 and 12′ may be configured generally similar to one another, and are associated with the same or different detector units at the detection system 18. The multiple (at least two) measured data pieces obtained from the light output of the light guides, respectively, are processed by the control unit 22. The at least two light guides may be used for measurement of the same or different parameters/conditions of the object.

For example, the system may be configured for measuring the individual's blood flow velocity and volume, using the light guide sensor carried by a fabric warned by the individual. The two light guides (fibers) 12 and 12′ are positioned such that they intersect the blood artery axis BA and are spaced from one another a certain distance d. The light input ports 12A and 12A′ of the fibers receive input light from the same light source unit (or separate light source units, as the case may be), and light output ports 12B and 12B′ of two fibers 12 and 12′ are associated with/connected to separate interferometric detector at the detection system 18. Two measured data pieces MD and MD′, indicative of light interaction with the body at different locations L and L′ respectively, are thus provided and processed by the control unit 22. The control unit 22 is configured for processing and analyzing the measured data pieces and determining the time that the same heart beat progresses the distance d from one fiber (location L) to the other fiber (location L′), and extracting the arriving modulated signal to each one of these locations, to determine the blood flow velocity and volume.

The following are some specific but not limiting examples of the configuration and operation of the optical sensing system of the invention. To facilitate illustration and understanding, the same reference numbers are used for identifying components that are common in all the examples.

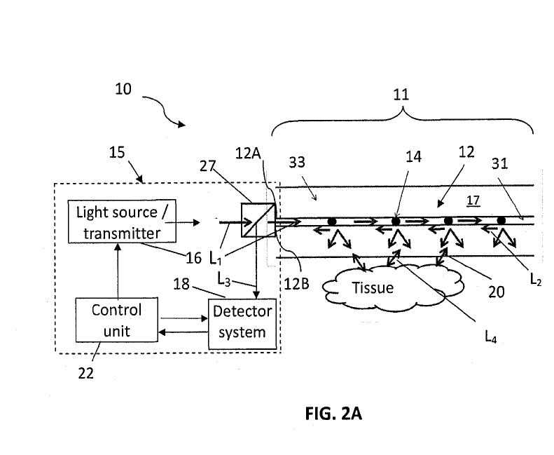

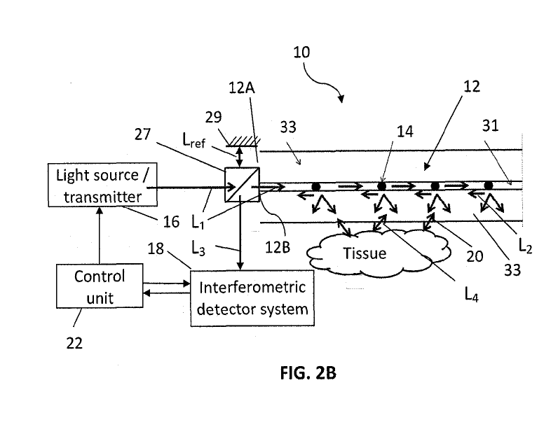

FIGS. 2A and 2B show schematically an optical sensing system 10 according to somewhat different examples of the invention adapted for monitoring one or more parameters or conditions of an object. The optical sensing system 10 includes an optical sensor device 12 including an elongated light guide 12 defining a cavity 17 for light propagation therethrough along the light guide (light propagation path), and having a light input port 12A at one end of the light guide, and one or more light output ports 12B.

In the present examples, the light guide 12 is an optical fiber caving a core 31 and cladding 33. Also, in the present not limiting examples of FIGS. 2A and 2B, the light output port 12B is located at the same end of the light guide as the input port 12A. In the example of FIG. 2B, the light detection system is configured as an interferometric light detector.

As shown, input light L₁ is injected from a light source/transmitter unit 16 into the light guide 12 at the light input 12A, e.g. via beam splitter 27 (the provision of which is optional), and propagates through the light guide cavity 17 along the light propagation path in the forward direction (input light propagation direction). In some embodiments, e.g. those where the detector-related light output and the light input are located at the same end of the light guide, the light guide is formed with light redirecting elements 14 arranged in a spaced-apart relationship along the light propagation path. The light redirecting elements may be implemented as scattering points in a fiber (e.g. specifically introduced defects), which reflect input light L₁ to cause reflected light L₂ to propagate back along said path towards the light output 12B, where it is collected and directed (e.g. via beam splitter 27) to the detector unit 18 (interferometric detector system in the example of FIG. 2B).

Generally, the detector system 18 may be of any known suitable configuration, being operable for continuously detecting light output from the light guide 12 during a predetermined time interval (measurement session), and generating output data in the form of a time function of the detected light. The detected light is modulated by interaction of light with the object. As indicated above, this may be direct interaction, or interaction via deformation of the light guide due to the motion originated in the object.

Considering the example of FIG. 2A, the modulation of the detected light may be amplitude modulation, which may be a direct measure of the motion pattern, and/or the modulation may be indicative of a change of polarization state of light, i.e. a so-called “polarization sensing”. In the latter case, light L₁ injected in the light guide 12 may be polarized light, and the system may include polarizers at the illumination path (input light propagation from the light source to the light input port 12A) and a detection path (light propagation path from the light output 12B to the detector system).

In the example of FIG. 2B, the interferometric detector system 18 is used which may have any known suitable configuration. In some embodiments, detected combined light L₃ is formed by output light L₂ (modulated by interaction with the object/tissue) interfering with a reference beam L_{ref} whose propagation path is varied using a mirror 29. In some other embodiments combined light L₃ is a result of interference between different components of the output light L₂, which are reflected from different locations along the light guide 12 such that they include light components modulated by interaction with a region of interest in the tissue (object) and non-modulated light components.

It should be understood that each of FIGS. 2A and 2B actually illustrates two examples of the invention, which may be implemented separately or in combination.

According to one example, there are no other light outputs in the light guide, other than light output port 12B where the output light is collected to the detector, and the interaction between light and tissue is via the flexible light guide 12. More specifically, due to the movement of the tissue (or, generally, motion/vibration originated at the tissue), the flexible light guide 12, being in physical contact with the tissue along its length or at least part thereof, deforms such that a deformation pattern of the light guide corresponds to the motion pattern of the tissue. The deformation of the light guide (cavity 17) results in the respective deformation of the light path in the cavity, i.e. trajectory of light L₂, and accordingly induces a modulation pattern, corresponding to the tissue motion. This modulation is identified in the detected signal/measured data (e.g. interference signal) at the detector system.

According to the other example shown in each of FIGS. 2A and 2B, the light guide 12 (which may not be flexible) is located in the close proximity of the tissue, and is formed with additional light output ports, i.e. interaction ports 20, arranged in a spaced-apart relationship along the light guide. Input light L₁ (and possibly also output light L₂ deflected by redirecting elements 14) propagates through the light guide 12, and portions L₄ thereof emerge from the light guide 12 through the interaction ports 20, interact with the tissue and return back into the light guide (e.g. using additional external re-directing elements on the outer surface of the light guide, which are not specifically shown). These light portions L₄ are therefore modulated by direct interactions with the tissue, and this modulation pattern corresponds to the tissue motion pattern.

As further shown in the figures, the optical sensing system 10 is associated with the electronic unit 15 including a control unit 22, which is connectable (via wires or wireless signal transmission of any known suitable type) with the detector system 18 for receiving and analyzing the detected signals (measured data) to determine one or more parameters of the tissue from the identified motion pattern originated in the tissue.

As further shown in the figures, in some embodiments, the control unit 22 may be appropriately connectable with the light source unit 16 and adapted to modulate the input light L₁, e.g. induce spectral modulation. Also, as described above, at least the fiber part 12 (optical sensor device), or both the fiber part 12 and the connector part 15 (electronic unit) may be configured to be disposable.

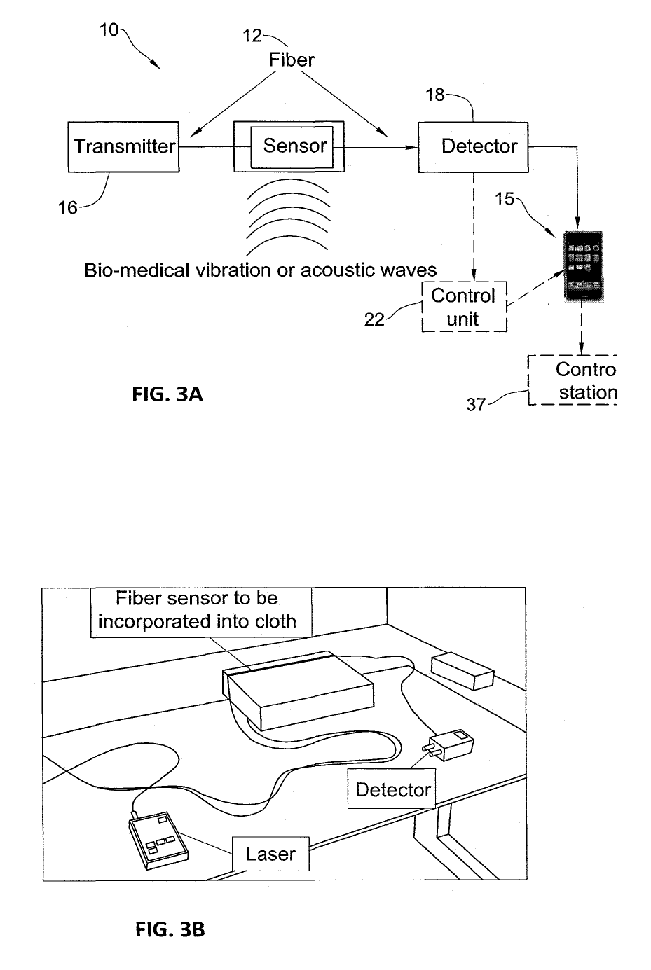

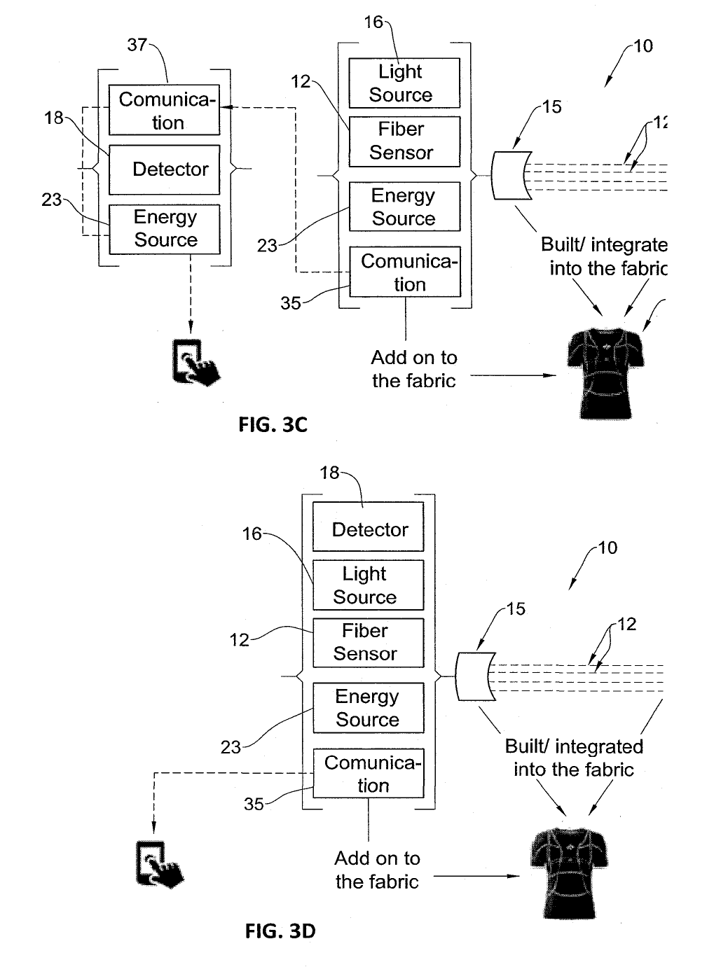

Reference is now made to FIGS. 3A-3D which illustrate a specific example of the optical sensing system of the invention. FIG. 3B shows in a self-explanatory manner a snapshot of the basic experimental system. FIG. 3A shows more specifically the configuration and operation of basic optical setup, including an optical sensor device 12 configured as described above according to either one or combination of the above-described embodiments, a transmitter (light source) 16, and a detector 18 (e.g. an interferometric detector). In this example the light guide sensor 12 (e.g. fiber based) extends between the transmitter 16 and detector 18, along the tissue being monitored, while being in the proximity of the tissue (thus including interaction ports 20) or in physical contact with the tissue (thus either including the interaction ports 20 or not). As also shown in the figure, the detector system 18 is in wireless communication with an external electronic device 15, such as phone device. Such electronic device 15 may be installed with data processor utility (control unit 22) for processing the detected signal, or, as shown in the figure in dashed lines, the phone device may be used just for transmitting the signal received from a stand-alone control unit 22 to a remote control station (server) 37 via a communication network, or a so-called “distributed data processing” may be used, e.g. the electronic device performs the initial processing and selectively forwarding data to the central station only upon identifying a certain degree of abnormality in the detected parameter/condition of the tissue.

FIGS. 3C and 3D present two alternatives for usage/integration of the fiber based optical sensor 12 into the fabric, utilizing partially and full integration of the system. In both examples, the optical sensing system 10 includes an optical sensor device (fiber-part 12) formed by multiple fibers (light guides), and a connector part 15 including an electronic system. The fiber-part 12 of the system is fully embedded in a fabric 40.

As for the electronic system 15, in some embodiments exemplified in FIG. 3D, it may be also fully integrated in the fabric 40, and may be operable (actuated) from a remote station via a communication utility 35 in the embedded electronic system 15. In such fully-embedded optical sensing system 10 exemplified in FIG. 3D, it includes the fiber sensor 12 and the electronic system 15 including a detection system 18, a light source unit 16; a power supply (battery) 23, and a communication utility 35, and may or may not include the processor (22 in FIG. 1) and may communicate either the processing results to an external control station/storage device or may communicate raw data (measured data) to an external control station to be processed and stored there.

As exemplified in FIG. 3C, the optical sensing system may include an embedded part and an external part. The embedded part may include the fiber part (fiber sensor) 12 and a part of the electronic system 15 including a light source 16, a poser supply 23, and a communication utility 35; and the external part includes a corresponding communication utility 37, detector 18 and energy source 23. Similarly, the system may include the processor 22 located in its external part and connected to the output of the detector and operable to communicate the processing results to an external control station/storage device, or may be configured for communicating with the processor located at the external control station. As indicated above, the software modules of the control unit/processor may be distributed between the embedded and external parts of the electronic system.

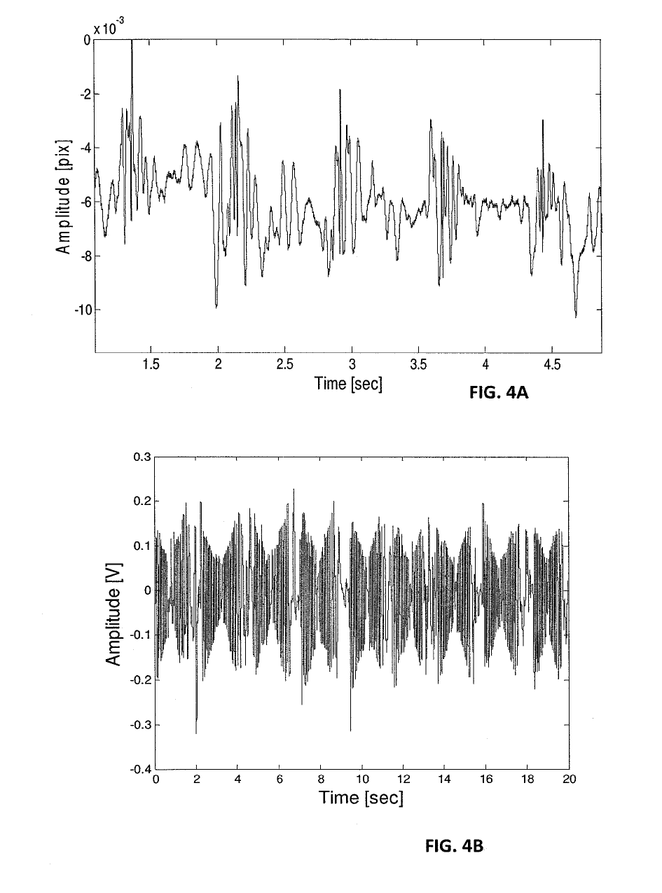

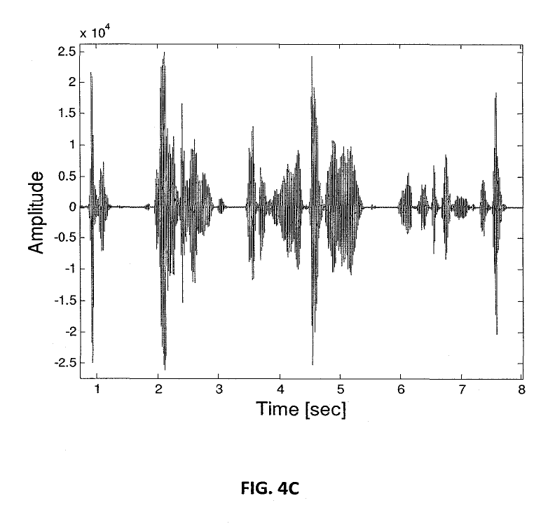

Preliminary experimental results for bio-medical non-contact fiber based sensing can be seen in FIGS. 4A-4C. For instance FIG. 4A shows the non-contact extraction of heart beating which yields typical beating rate of 1.12 Hz and it exactly matches the reference measurement of 67 bpm measured with electric Mio watch. FIG. 4B shows extraction of breathing, and FIG. 4C shows that the invention can also be used as a microphone that can record the voice of the speaker or the sounds around him.

1. An optical sensing system for monitoring one or more parameters or conditions of an object, the optical sensing system comprising: an optical sensor unit comprising at least one elongated light guide configured for placing in proximity of the object, the light guide defining a cavity for light propagation therethrough along a light propagation path, and having a light input port and at least one light output port; and a detector system for receiving light propagating from the at least one light output port, the detector system being configured and operable for monitoring a signal modulated by light interaction with the object and being indicative of the at least one parameter/condition of the object.

2. The optical sensing system according to claim 1, wherein the detector system is configured as an interferometric detector system adapted for monitoring an interference signal resulting from interference between light modulated by the interaction with the object and non-modulated light, the interference signal being indicative of the at least one parameter of the object.

3. The optical sensing system according to claim 2, wherein the non-modulated light is light propagating along a reference path in the interferometric detector system outside the light guide.

4. The optical sensing system according to claim 2, wherein the interference signal is indicative of self-interference between the modulated and non-modulated light components propagating in said cavity and being reflected from different locations along the light guide being respectively affected and non-affected by an external signal originated at the object.

5. The optical sensing system of any one of the preceding claims, wherein the signal modulated by the light interaction with the object is indicative of motion originated at the object, thereby enabling monitoring acoustic signals corresponding to the motion originated at the object.

6. The optical sensing system according to claim 1, wherein the light guide as at least one of the following configurations:
(i) the light guide comprises one or more interacting ports located in said cavity downstream of the input port of the light guide with respect to a direction of propagation of the input light, the interacting port being configured to allow light propagating in the light guide to emerge from the light guide towards the object and receive light returned from the object and being modulated by direct interaction with the object;
(ii) the light guide comprises one or more light redirecting elements located in one or more locations, respectively, in the cavity defined by the light guide and adapted for directing light, propagating in said cavity, towards the one or more output ports;
(iii) the light guide is substantially flexible allowing its placing in contact with and along a portion of the object;
(iv) the light guide comprises at least one optical fiber.

7-8. (canceled)

9. The optical sensing system of claim 1, wherein the light guide is substantially flexible allowing its placing in contact with and along a portion of the object, interaction between the flexible light guide and the portion of the object causes deformation of the light guide according to a motion originated at the object, thereby modulating light propagating along said path, such that a light modulation pattern is indicative of a deformation pattern of the light guide which corresponds to a motion pattern of the object.

10. (canceled)

11. The optical sensing system of claim 1, wherein the light guide comprises at least one optical fiber, the optical fiber being formed with one or more scattering points arranged in one or more locations a core of the fiber, the one or more scattering points directing light propagating in the fiber towards the one or more output ports of the fiber.

12. The optical sensing system of claim 1, wherein the detection system comprises a communication utility adapted for data communication with a control unit, which is adapted for processing and analyzing the signal modulated by the light interaction with the object and determining said one or more parameters of the object.

13. (canceled)

14. The optical sensing system of claim 1, further comprising a light source unit for producing input light and directing the produced light into the light guide via the input port thereof.

15. The optical sensing system of claim 14, wherein the light source unit has at least one of the following configurations: (1) the light source unit is configured and operable for producing the input light having light components of different wavelengths; and (2) the light source unit is configured and operable for producing light of predetermined polarization state.

16-19. (canceled)

20. The optical sensing system claim 1, having at least one of the following configurations: (a) the input and output ports are associated with the same end of the elongated light guide; (b) the input and output ports are associated with opposite ends of the elongated light guide.

21. (canceled)

22. The optical sensing system of claim 1, wherein the optical sensor unit comprises at least one additional light guide having a light input port and at least one light output port, said detector system comprising at least two detectors associated with the at least two light guides respectively, each of the at least two detectors receiving light output from the respective light guide and generating measured data indicative of light interaction with the object at a location of the respective light guide, the detection system being configured for communication with a control unit adapted for processing and analyzing the measured data and determining said one or more parameters of the object.

23. (canceled)

24. An optical sensor device for use in a sensing system for monitoring one or more parameters or conditions of an object, the device comprising: a light guide unit comprising at least one elongated light guide configured for placing in proximity of an object to be monitored, the light guide defining a cavity for light propagation therethrough along a light propagation path, and having a light input port for inputting light to propagate along said path, and at least one output port associated with a detection system, said elongated light guide having at least one of the following configurations:
(i) the at least one light guide comprises one or more interacting ports located downstream of the input port, the interacting port being configured to allow light to emerge from the light guide towards the object and receive light returned from the object and being modulated by a modulation pattern indicative of interaction of light with the object, said modulation pattern corresponding to a motion pattern originated at the object, being indicative of at least one parameter or condition of the object; and
(ii) the at least one light guide is substantially flexible allowing its placing in contact with and along a portion of the object, such that interaction between the flexible light guide and the portion of the object causes deformation of the light guide according to a motion originated at the object, thereby modulating light propagating along said path, such that a light modulation pattern is indicative of a deformation pattern of the light guide which corresponds to a motion pattern originated at the object being indicative of at least one parameter or condition of the object.

25-26. (canceled)

27. The optical sensor device of claim 24, comprising an interferometric detector located at said at least one output of the light guide for detecting an interference signal resulting from interference between the light modulated by the interaction with the object and non-modulated light, the interference signal being indicative of the at least one parameter or condition of the object.

28. The optical sensor device of claim 27, wherein the non-modulated light is light propagating along a reference path in the interferometric detector outside the light guide.

29. The optical sensor device of claim 27, wherein the interference signal is indicative of self-interference between the modulated and non-modulated light components propagating in said cavity and being reflected from different locations along the light guide being respectively affected and non-affected by an external signal originated at the object.

30. The optical sensor device of claim 24, wherein the light guide has one of the following configurations: (i) the light guide comprises light directing elements located in a spaced-apart arrangement in the cavity defined by the light guide and adapted for direct light, propagating in said cavity, towards the at least one output port; (ii) the light guide comprises at least one optical fiber, the optical fiber being formed with an array of scattering points arranged in spaced-apart relationship along a core of the fiber, said scattering points directing light propagating in the fiber towards the at least one output port.

31. (canceled)

32. The optical sensor device of claim 24, having at least one of the following configurations: (1) the input and output ports are associated with the same end of the elongated light guide; (2) the input and output ports are associated with opposite ends of the elongated light guide.

33-34. (canceled)

35. The optical sensor device of claim 24, wherein the light guide unit comprises at least one additional light guide having a light input port and at least one light output port.

36. A fabric material carrying the optical sensor system of claim 1, being integral with or embedded in the fabric material.

37. A fabric material carrying the optical sensor device of claim 24, being integral with the fabric material.

38. The fabric material of claim 36, configured to be worn by an individual for carrying out one or more of the following: non-contact bio monitoring of one or more parameters of the individual comprising at least one of the following: breathing, heart beating, blood pulse pressure, pulse oximetry related parameters, lactate concentration, blood flow velocity, blood volume; and recording acoustic signals indicative of conversations performed by the individual in a range of up to a few meters from the fabric material.

39. A fabric material of claim 37, configured to be worn by an individual for carrying out one or more of the following: non-contact bio monitoring of one or more parameters of the individual comprising at least one of the following: breathing, heart beating, blood pulse pressure, pulse oximetry related parameters, lactate concentration, blood flow velocity, blood volume; recording acoustic signals indicative of conversations performed by the individual in a range of up to a few meters from the fabric material.

40. (canceled)

41. The fabric material of claim 36, wherein the optical sensor device comprises a plurality of at least two of the light guides located in a spaced apart relationship such that when the fabric material is warned by an individual, the at least two light guides are positioned in different spatial locations along the same blood artery.
